(19) United States
(12) Patent Application Publication
(10) Pub. No.: US 2022/0023669 A1
(43) Pub. Date: Jan. 27, 2022

(54) HEPATIC ULTRASOUND IMPROVES METABOLIC SYNDROME, FATTY LIVER DISEASE AND INSULIN RESISTANCE AND DECREASES BODY WEIGHT

(71) Applicant: THE FEINSTEIN INSTITUTES FOR MEDICAL RESEARCH, Manhasset, NY (US)

(72) Inventors: Tomas Huerta, Brooklyn, NY (US); Sangeeta S. Chavan, Syosset, NY (US); Kevin J. Tracey, Old Greenwich, CT (US)

(73) Assignee: THE FEINSTEIN INSTITUTES FOR MEDICAL RESEARCH, Manhasset, NY (US)

(21) Appl. No.: 17/293,522

(22) PCT Filed: Nov. 25, 2019
(86) PCT No.: PCT/US2019/062960

§ 371 Date: May 13, 2021

Related U.S. Application Data
(60) Provisional application No. 62/771,943, filed on Nov. 27, 2018.

(51) Int. Cl. A61N 7/02

(52) U.S. Cl. CPC A61N 7/02; A61N 2007/0008

(57) ABSTRACT

Methods are disclosed for treating a subject with metabolic syndrome, fatty liver disease, insulin resistance, inflammation or elevated body weight using hepatic ultrasound.

### CROSS-REFERENCE TO RELATED APPLICATIONS

This application claims the benefit of U.S. Provisional Patent Application No. 62/771,943, filed on Nov. 27, 2018, the contents of which are herein incorporated by reference into the subject application.

### BACKGROUND OF THE INVENTION

Throughout this application various publications are referred to in parentheses. Full citations for these references may be found at the end of the specification. The disclosures of these publications are hereby incorporated by reference in their entirety into the subject application to more fully describe the art to which the subject invention pertains.

Metabolic syndrome is a cluster of metabolic disorders, which cumulatively increase the risk for cardiovascular disease, fatty liver disease and type 2 diabetes. Metabolic syndrome is highly prevalent in the United States, affecting approximately 25% of the population. The major risk factors for metabolic syndrome are abdominal obesity, dyslipidemia, high blood pressure and high fasting blood sugar. To be diagnosed with metabolic syndrome, one must have at least three of these risk factors (1). Two factors that are referred to as the underlying causes for metabolic syndrome are abdominal obesity and insulin resistance (2).

Abdominal adiposity is the most prevalent manifestation of metabolic syndrome. There is evidence that implicates dysregulated fatty acid metabolism contributes to an inulin resistant state in individuals with excess visceral obesity. Increased presence of fatty acid flux to the liver may impair liver metabolism, increasing hepatic glucose production. The protein adiponectin, found in high concentrations in individuals with a healthy metabolic state, is known to decrease in patients with visceral obesity. Adiponectin has been found to have several effects in vitro that are associated with healthy insulin signaling, a key issue for obese patients. Visceral obesity has also been linked to elevated CRP concentrations, associated with increased proinflammatory cytokine levels, specifically TNF-α and IL-6. Studies have associated macrophages in the white adipose tissue to a sustained low-grade inflammatory response (3). Another hypothesis posits that visceral fat accumulation is a reflection of the subcutaneous adipose tissue to store fats as an energy sink. Following this hypothesis, the deficit in the capacity of subcutaneous fat to store energy results in accumulation of fat in less efficient sites including the liver, heart, skeletal muscle and pancreatic B-cells. This process is referred to as ectopic fat deposition. These findings and this hypothesis point to visceral obesity as a major contributing factor to diminished metabolic health, and a key component to metabolic syndrome (4).

Insulin is an important metabolic hormone that aids the body in regulating the levels of glucose. Insulin resistance a condition where cells in the body that usually respond to insulin, in the muscle, fat, and liver, become insensitive to the hormone. Insulin resistance is a key component of metabolic syndrome, and is a known precursor to the development of type 2 diabetes. When a patient is insulin resistant, their body will produce greater amounts of insulin, known as hyperinsulinemia, which is ineffective at reducing the patient's chronically high blood glucose levels (5).

Currently, there is a dearth in pharmacological treatments for metabolic syndrome. The standard of care is commonly limited to lifestyle modification and change in diet (6). For patients diagnosed as morbidly obese, bariatric surgery is considered as a means of treatment. For patients diagnosed with type 2 diabetes, doctors regularly prescribe medications such as metformin, and insulin therapy. The shortage of treatment options reflects a lack of understanding of the pathological mechanisms involved in the development of metabolic syndrome.

There is a growing body of literature in the field of neuromodulation that puts forth focused ultrasound as a novel noninvasive methodology to stimulate neurons in the brain and the periphery. Focused ultrasound has been shown to be relatively innocuous to the body, with a large window of safe stimulation, prior to seeing any heat effects or tissue damage. Although the exact mechanism by which focused ultrasound induces neuronal firing is still under investigation, the utility of a noninvasive and safe method for stimulating neurons in target organs provides an exciting therapy (7).

Recent studies have demonstrated the utility of using high-intensity focused ultrasound stimulation as a novel noninvasive stimulation strategy in the periphery (8). Stimulation of the nerve plexus in the liver, known as the porta hepatis, was shown to reduce blood glucose rise in rats in an acute endotoxemia experiment. It was found that stimulation of the porta hepatis, specifically, was required to induce this blood glucose suppression, whereas stimulation of a non-heavily innervated lobe of the liver was found to be insufficient to control the blood glucose response. This result suggests that the nervous system is linked to the effect that high-intensity focused ultrasound stimulation has on an aspect of metabolic control.

The present invention addresses the need for improved methods for treating metabolic syndrome, fatty liver disease and insulin resistance, by using high-intensity focused ultrasound stimulation of the porta hepatis.

### SUMMARY OF THE INVENTION

Method are disclosed for treating metabolic syndrome, treating fatty liver disease, improving insulin resistance, treating inflammation and decreasing body weight in a subject in need thereof comprising applying hepatic ultrasound to the subject in an amount effective to one or more of treat metabolic syndrome, treat fatty liver disease, improve insulin resistance, treat inflammation and decrease body weight.

### BRIEF DESCRIPTION OF THE DRAWINGS

FIG. 1A. High intensity focused ultrasound reduces body weight. Mice were given either high fat high carbohydrate (HFHC) diet (upper plots) or low fat diet (LFD) control (lower plots) for a period of 9 weeks. On week 9, the mice either received high intensity focused ultrasound (HIFU) stimulation (closed circles) or sham stimulation (open circles) for the remainder of the experiment. Starting at week 12, the HFHC-HIFU group (closed circles on upper plot) had significantly attenuated body weight in comparison to HFHC-sham controls (**p<0.01, 2-way ANOVA, week 12 HFHC-HIFU vs HFHC-sham).

FIG. 1B. Mice on different diets have similar food intake prior to ultrasound stimulation. The food intake of the mice was monitored and calculated per cage per week. No significant difference was found among any of the groups in the pre-stimulation period (p>0.05, one-way ANOVA, weeks 1-8).

FIG. 1C. Hepatic HIFU stimulation reduces food intake in high fat high carbohydrate fed mice during the stimulation period.

FIG. 1D. Hepatic HIFU stimulation reduces visceral fat accumulation. Fat resected post-mortem from three areas (epididymal, retroperitoneal/perirenal, and mesenteric) was weighed and compared between the groups. The HFHC-HIFU group has significantly lower fat accumulation than the HFHC-sham group for each fat pad (****p<0.0001, 2-way ANOVA, epididymal and mesenteric, HFHC-HIFU vs HFHC-sham; ***p<0.001, 2-way ANOVA, retroperitoneal/perirenal, HFHC-HIFU vs HFHC-sham). From left to right in each tissue group: LFD-sham, LFD-HIFU, HFHC-sham, HFHC-HIFU.

FIG. 2A. Hepatic HIFU stimulation reduces circulating blood glucose levels. Serum collected from week 9 and 16 was assessed for circulating blood glucose by Freestyle InsuLinx Blood Glucose Monitoring System. HFHC-HIFU mice had significantly reduced levels of blood glucose in response to ultrasound stimulation (**p<0.01, 2-way ANOVA, week 9 HFHC-HIFU vs HFHC-HIFU week 16, n=14 per group).

FIG. 2B. Hepatic HIFU stimulation reduces insulin levels. Serum collected from week 9 and 16 was assessed for circulating insulin levels using a MILLIPLEX Metabolic Hormone Magnetic Bead Panel Multiplex Assay (Millipore Sigma). HFHC-HIFU mice had significantly reduced levels of blood glucose in response to ultrasound stimulation (**p<0.01, 2-way ANOVA, week 9 HFHC-HIFU vs HFHC-HIFU week 16, n=10 per group).

FIG. 2C. Hepatic HIFU alleviates insulin resistance in HFHC diet fed mice. The HOMA-IR formula [fasting serum glucose×fasting serum insulin/22.5] was used to assess the insulin resistance of HFHC diet fed mice at week 16. Hepatic HIFU stimulation was shown to reduce the insulin resistance of HFHC-HIFU mice when compared to HFHC-sham mice (***p<0.001, HFHC-sham vs HFHC-HIFU week 16, 1-way ANOVA, n=10 per group).

FIG. 2D. Hepatic HIFU stimulation improves performance in the glucose tolerance test. On week 16, fasted mice were subjected to a glucose tolerance test (GTT, 1 mg/kg, I.P.). HFHC-HIFU mice demonstrated increased tolerance to glucose challenge in comparison to HFHC-sham mice (*p<0.05, 2-way ANOVA, 15 min post-injection, HFHC-HIFU vs HFHC-sham; ****p<0.0001, 2-way ANOVA, 30, 45, 60 min, HFHC-HIFU vs HFHC-Sham; ***p<0.001, 2-way ANOVA, 90 min post-injection, HFHC-HIFU vs HFHC-sham, n=10 mice per group). Plots from top to bottom at 120 minutes: HFHC-sham, HFHC-HIFU, LFD-sham, LFD-HIFU.

FIG. 2E. Hepatic HIFU stimulation improves performance in the glucose tolerance test. Area under the curve for the GTT demonstrates the HFHC-HIFU mice have significantly improved glucose tolerance (**p<0.01, 2-way ANOVA, HFHC-HIFU vs HFHC-sham).

FIG. 3A. Hepatic HIFU stimulation reduces circulating resistin levels. Circulating levels of resistin in HFHC diet fed mice were collected at weeks 9 and 16, then measured using MILLIPLEX Metabolic Hormone Magnetic Bead Panel Multiplex Assay (Millipore Sigma). HFHC-sham mice did not demonstrate a significant difference between weeks 9 and 16, while HFHC-HIFU mice demonstrated a significant reduction in resistin levels (*p<0.05, 2-way ANOVA, week 9 HFHC-HIFU vs HFHC-HIFU week 16, n=10 per group).

FIG. 3B. Hepatic targeted HIFU stimulation attenuates leptin increase in HFHC diet fed mice. Mice on a HFHC diet were assessed for their circulating serum leptin levels between weeks 9 and 16. The HFHC-sham group had significantly increased leptin levels, while HFHC-HIFU mice did not significantly change between weeks 9 and 16 (*p<0.05, 2-way ANOVA, week 9 HFHC-sham vs HFHC-sham week 16, n=10 per group).

FIG. 3C. Hepatic targeted HIFU stimulation attenuates adiponectin decrease in HFHC diet fed mice. Mice on a HFHC diet were assessed for their circulating serum adiponectin levels between weeks 9 and 16. The HFHC-sham group had significantly lowered adiponectin levels. This decrease was attenuated in HFHC-HIFU mice between weeks 9 and 16 (*p<0.05, 2-way ANOVA, week 9 HFHC-sham vs HFHC-sham week 16, n=10 per group).

FIG. 3D. Hepatic targeted HIFU stimulation attenuates cholesterol increase in HFHC diet fed mice. Cholesterol levels from HFHC diet fed mice were evaluated between weeks 9 and 16. The HFHC-sham group had significantly increased cholesterol levels, while this increase was attenuated in HFHC-HIFU mice between weeks 9 and 16 (*p<0.05, 2-way ANOVA, week 9 HFHC-sham vs HFHC-sham week 16, n=10 per group).

FIG. 3E. Hepatic targeted HIFU stimulation attenuates triglyceride increase in HFHC diet fed mice. Triglyceride levels from HFHC diet fed mice were evaluated between weeks 9 and 16. The HFHC-sham group had significantly increased triglyceride levels, while there was a significant decrease of triglycerides in the HFHC-HIFU mice between weeks 9 and 16 (**p<0.01, 2-way ANOVA, week 9 HFHC-sham vs HFHC-sham week 16, n=10 per group; *p<0.05, 2-way ANOVA, week 9 HFHC-HIFU vs HFHC-HIFU week 16, n=10 per group).

FIG. 4A. Hepatic targeted HIFU stimulation attenuates alanine aminotransferase increase in HFHC diet fed mice. Alanine aminotransferase (ALT) levels were evaluated in HFHC diet fed mice between weeks 9 and 16. The HFHC-sham group had significantly increased ALT levels. This increase was attenuated in HFHC-HIFU mice between weeks 9 and 16 (*p<0.05, 2-way ANOVA, week 9 HFHC-sham vs HFHC-sham week 16, n=10 per group).

FIG. 4B. Hepatic HIFU stimulation reduces the severity of inflammatory cell infiltration in the liver. H&E staining of liver sections from HFHC diet fed mice were assessed for mean inflammation, which was calculated by scoring amount of inflammatory clusters within 5 fields of view per section. The histological assessment revealed that the HFHC-HIFU group had lower severity of inflammatory cell infiltration into the liver when compared to HFHC-sham mice. The HFHC-HIFU group did not have any sections that had a “severe” (score of 3), mean inflammation score, whereas over 25% of the sections from HFHC-sham mice received a score of 3.

FIG. 4C. Representative slides of H&E stained liver sections. Liver sections were processed by H&E staining and underwent histological assessment by a trained pathologist. The top (20×) and bottom (40×) left panels show a LFD-sham slide with preserved hepatocyte morphology, no steatosis and no inflammatory cell clusters. The top (20×) and bottom (40×) middle panels show a slide from the HFHC-sham group, which has steatosis (white arrow), and inflammatory clusters (black arrow). The top (20×) and bottom (40×) right panels show a slide from the HFHC-HIFU group, which has steatosis (white arrow), but no inflammatory clusters.

### DETAILED DESCRIPTION OF THE INVENTION

The present invention provides a method for one or more of treating metabolic syndrome, treating fatty liver disease, improving insulin resistance, treating inflammation and decreasing body weight in a subject in need thereof comprising applying hepatic ultrasound to the subject in an amount effective to one or more of treat metabolic syndrome, treat fatty liver disease, improve insulin resistance, treat inflammation and decrease body weight.

As used herein, to treat a disease or condition means to alleviate a sign or symptom of the disease or condition.

The key sign of metabolic syndrome is central obesity, also known as visceral, male-pattern or apple-shaped adiposity. It is characterized by adipose tissue accumulation predominantly around the waist and trunk. Other signs of metabolic syndrome include high blood pressure, decreased fasting serum HDL cholesterol, elevated fasting serum triglyceride level, impaired fasting glucose, insulin resistance, and prediabetes.

Preferably, hepatic ultrasound reduces one or more of the subject's food intake, visceral fat accumulation and body weight.

Preferably, hepatic ultrasound does one or more of reduce blood glucose levels, reduce insulin levels, improve insulin resistance, and improve glucose tolerance in the subject.

Preferably, hepatic ultrasound reduces blood levels of one or more of resistin, leptin, cholesterol, triglyceride and alanine aminotransferase in the subject.

Preferably, hepatic ultrasound increases blood levels of adiponectin in the subject.

Preferably, the fatty liver disease is nonalcoholic fatty liver disease. The fatty liver disease can be nonalcoholic steatohepatitis.

In one embodiment, the subject is on a high-fat, high-carbohydrate diet.

Ultrasound devices typically operate with frequencies from 20 kHz up to several gigahertz. Preferably, the ultrasound is high intensity focused ultrasound (HIFU). For example, a 1.1 MHz HIFU transducer (e.g., Sonic Concepts H106) can be used. The transducer can have, for example, a 70 mm diameter.

Stimulus parameters can be readily optimized by one of ordinary skill in the art. For example, in a previous study (8), stimulation parameters were tested for the optimal nerve stimulation in rodent peripheral end organs. The input volts (Vpeak) was varied from 0.5 to 62 volts, with corresponding peak pressure changes ranging from 0.01 to 1.72 MPa. The maximal response was found between 0.83 and 1.27 MPa delivered peak positive pressure. The pulse repetition period was tested at 0.5 ms, 200 ms, and 1000 ms. With the maximal effect found at 200 ms. Pulse length was also studied in a range from 18.18 to 1363.63 μs, with a maximal response found between 136.36 and 227.27 μs.

Preferably, the ultrasound targets the porta hepatis nerve plexus in the liver. Imaging of the subject can be used to identify the target location prior to application of HIFU.

The subject can be any mammal and is preferably a human.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

Animals. Experiments were performed on male C57BL/6J mice (8 weeks old, Jackson Lab, Bar Harbor, Me., USA). All procedures performed on the mice were in accordance with National Institutes of Health (NIH) Guidelines under protocols approved by the Institutional Animal Care and Use Committee (IACUC) of the Feinstein Institutes for Medical Research, Northwell Health, Manhasset, N.Y. USA.

Experimental Design. 6-8 week old C57BL/6J mice were obtained from The Jackson Laboratory. The mice were fed regular chow for 10 days in a reverse light cycle room, and then switched to a high-fat diet (D12492, 60% kcal from fat), or its corresponding isocaloric low-fat diet (10% kcal from fat) for 16 weeks. Mice in the high-fat group received sugar supplemented water (55% Fructose, 45% Sucrose); thus, they were on a high-fat high-carbohydrate (HFHC) dietary model. After 8 weeks, the HFHC mice were divided into two groups, either treated with high intensity focused ultrasound (HIFU) stimulation of the porta hepatis (once daily), localized using an ultrasound imaging probe, or sham stimulation for the following 8 weeks. After 8 weeks, the low-fat control diet mice were treated with either the HIFU stimulation or the sham stimulation for the remaining 8 weeks. Body weight and food intake for all the mice were monitored on a weekly basis. At the end of the experiment, mice were euthanized and liver weight, visceral adipose weight, cytokine and adipokine levels, metabolic profile, insulin levels, and liver histology were evaluated. Prior to euthanasia, mice were subjected to a glucose tolerance test.

Blood Glucose Determination. Blood glucose levels were assessed weekly by cheek bleed and using a Freestyle blood glucose monitoring system (Abbott Diabetes Inc., Alameda, Calif., USA) with Freestyle blood glucose strips following the manufacturer's recommendations. Mice were fasted 3 hours prior to blood glucose assessment. After blood collection the mice were given a 100 μL injection of saline IP.

Blood Collection and Tissue Harvesting. After a morning fast (3-4 hr) blood was collected weekly using the cheek bleed method. Approximately 300 μL of whole blood were sampled per animal. Blood samples were spun in a centrifuge (10 min at 5000 rpm, then 2 min at 10000 rpm) and the serum was extracted and frozen for further evaluation.

At the end of the study, mice were subjected to an overnight fast. After body weight measurement and blood glucose determination, and blood collection via cheek bleed, mice were euthanized by CO₂ asphyxiation. Mice were perfused with 4% PFA, then visceral adipose tissue and livers were rinsed with saline and weighed. The largest lobe of the liver was sectioned for H&E staining.

High Intensity Focused Ultrasound Stimulation. Mice were anesthetized at 2% isoflurane at 1 L/min O₂. Mice were then placed on a water circulating warming pad, with a rectal thermometer probe to maintain body temperature. The area above the stimulation target was shaved and hair was fully removed with Nair. The porta hepatis of the mice was localized using a custom ultrasound imaging device (GE Healthcare). The location was marked with a permanent marker and a focused ultrasound stimulation probe (GE Healthcare) was placed on the target area.

Function generator. A pulsed sinusoidal waveform was produced by an Agilent 33120A function generator. HIFU stimulation was carried out at a pulse center frequency of 1.1 MHz, with a pulse repetition period of 200 ms, at 0.27 duty cycle, and pulse length of 136.36 μs.

RE Power Amplifier and Matching Network. The signal from the function generator was routed to an ENI 350L RF power amplifier. The amplified signal from the RF Power Amplifier was routed to an impedance-matching network (set to 1.1. MHz), which was connected to the HIFU transducer.

HIFU Transducer. The transducer had a 70 mm diameter with a 65 mm radius of curvature, with a 20 mm diameter hole in the center. The depth of focus was 65 mm. The focal point had a full width at half amplitude of 1.8 mm laterally and 12 mm in depth. The HIFU transducer was coupled to the animal with a 6 cm tall plastic cone filled with degassed water.

Delivered Pressure. The estimated delivered ultrasound pressure was 0.83 MPa. This pressure was optimized for rodent end-organ stimulation in previous experiments (8). This acoustic pressure is well under the FDA limits for diagnostic imaging. Mechanical index was measured at 0.58, whereas the limit is 1.9. Thermal index was measured at 0.44, where the limit is 2.

Length of Stimulation. The hepatic portal of the mice was stimulated for 1 min, followed by a 30 sec rest, then 1 min of stimulation. The rest period was used to reduce the possibility of any heat effects from prolonged stimulation.

Serum Adipokine Determination and Other Blood Biochemistry Tests. Serum samples were centrifuged from whole blood drawn by cheek bleeding (10 min at 5000 rpm, then 2 min at 10000 rpm). The samples were then analyzed with a Millipore MILLIPLEX mouse adipokine panel assay for insulin, leptin, MCP-1, PAI-1, resistin, TNF, IL-6, glucagon, GLP-1, C-peptide, and ghrelin. Serum samples were assessed with a Piccolo Xpress chemistry analyzer using a Lipid Panel Plus: cholesterol, HDL, triglycerides, ALT, AST, glucose, nHDLc, total cholesterol/HDL, LDL, and VLDL. Serum adiponectin was measured by using a Mouse Adiponectin ELISA (Invitrogen, Carlsbad, Calif., USA) according to manufacturer's recommendations.

Insulin Resistance Evaluation. Glucose and insulin levels were utilized to determine insulin resistance by applying the homeostatic model assessment-insulin resistance (HOMA-IR) formula.

Liver Histology, Hepatic Steatosis, and Hepatic Inflammation Assessment. Livers were fixed by a perfusion of formalin, and then the largest lobe of the liver was imbedded in paraffin. The lobe was then sliced and the liver tissue sections were subjected to hematoxylin and eosin (H&E) staining. Microscope slides were then prepared. Hepatic steatosis and inflammation were semiquantified by microscopic evaluation by a blinded pathologist. The grading criteria for steatosis was: no fat accumulation (grade 0); less than 33% fat-containing hepatocytes (grade 1); less than 66% fat-containing hepatocytes (grade 2); more than 66% fat-containing hepatocytes (grade 3). This grading process was applied to microvesicular steatosis, and macrovesicular steatosis. Hypertrophy, defined as cellular enlargement of hepatocyte 1.5× the normal diameter, was also scored and graded as the percentage of the total area, similar to the aforementioned method. Inflammation was evaluated as the number of foci (cluster n>5) of inflammatory cells. Inflammation was assessed in 5 different fields at 100× magnification and the average was scored as normal (<0.5 foci), slight (0.5-1.0 foci), moderate (1.0-2.0 foci), and severe (>2.0 foci).

Glucose Tolerance Tests. At the end of the 16 week period, mice from the four experimental groups were subjected to a glucose tolerance test. The mice were fasted overnight (18 h), weighed, and injected with glucose (10% D glucose solution; Sigma, St. Louis, Mo., USA; 1 g/kg; I.P.). Glucose levels were determined at 0, 15, 30, 60, and 120 min after glucose administration in blood from the tail vein.

Statistical Analysis. Data are expressed as mean±SEM. Significant differences were assessed by using two-way analysis of variance (ANOVA). Differences with P<0.05 were considered statistically significant.

### Results

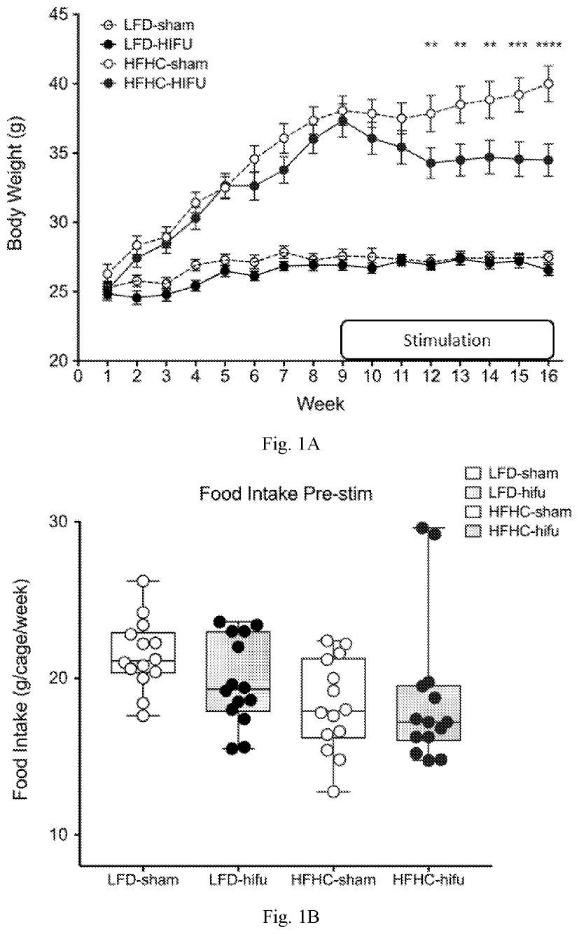

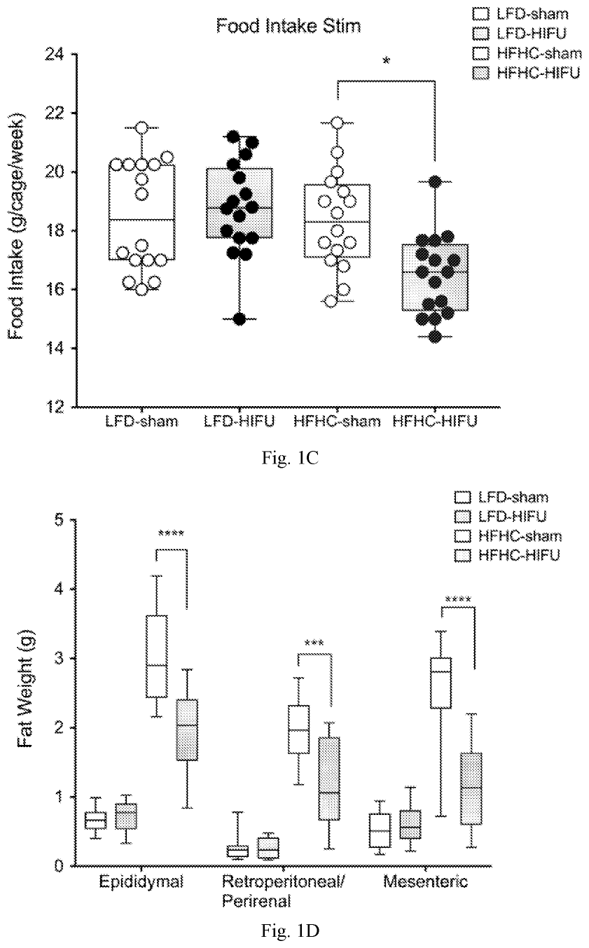

High Intensity Focused Ultrasound Reduces Body Weight Gain, Food Intake, and Abdominal Adiposity in High-Fat High-Carbohydrate Fed Mice. In order to assess the viability of high intensity focused ultrasound (HIFU) stimulation as a treatment for metabolic syndrome, mice were fed a high-fat high-carbohydrate (HFHC) diet for 9 weeks prior to stimulation. The mice on the HFHC diet gradually increased weight over the course of the first 9 weeks, which reached a difference of approximately 10 g (p<0.0001, 2-way ANOVA) compared to low-fat diet (LFD) fed control mice. Beginning at week 9, mice on both diets were separated into subgroups that received either daily HIFU stimulation targeted to the porta hepatis or sham stimulation for the remainder of the study (until week 16). Thus, the study contained four groups LFD-sham, LFD-HIFU, HFHC-sham, and HFHC-HIFU. Mice in the HFHC-sham stimulation group continued to increase in body weight from weeks 9-16, while the mice in the HFHC-HIFU stimulation group stopped gaining weight and gradually reduced body weight, reaching a significant difference with the HFHC-sham group by week 12 (FIG. 1A, p<0.01, 2-way ANOVA). There was no significant difference found between the LFD fed groups that either received HIFU stimulation or sham stimulation.

There was no significant difference found among the average food intake (g/cage/week) for the four groups prior to the stimulation period (FIG. 1B). However, the food intake of the HFHC-HIFU group was reduced in the post stimulation period when compared to the HFHC-sham group (FIG. 1C, p<0.05, 1-way ANOVA).

The abdominal adiposity of the mice was assessed post-mortem by harvesting three sites of fat tissue (mesenteric, retroperitoneal/perirenal, and epididymal). Mice in the HFHC-sham group had increased abdominal adiposity in all three of the fat deposits as compared with mice in the LFD-sham and LFD-HIFU groups. Mice in the HFHC-HIFU group had significantly reduced abdominal adiposity in the three fat pads when compared to HFHC-sham mice (FIG. 1D, p<0.01, 2-way ANOVA).

Together these results demonstrate that HIFU stimulation on HFHC fed mice attenuates the degree of body weight gain, average food intake, and fat pad accumulation that is seen in the HFHC-sham mice.

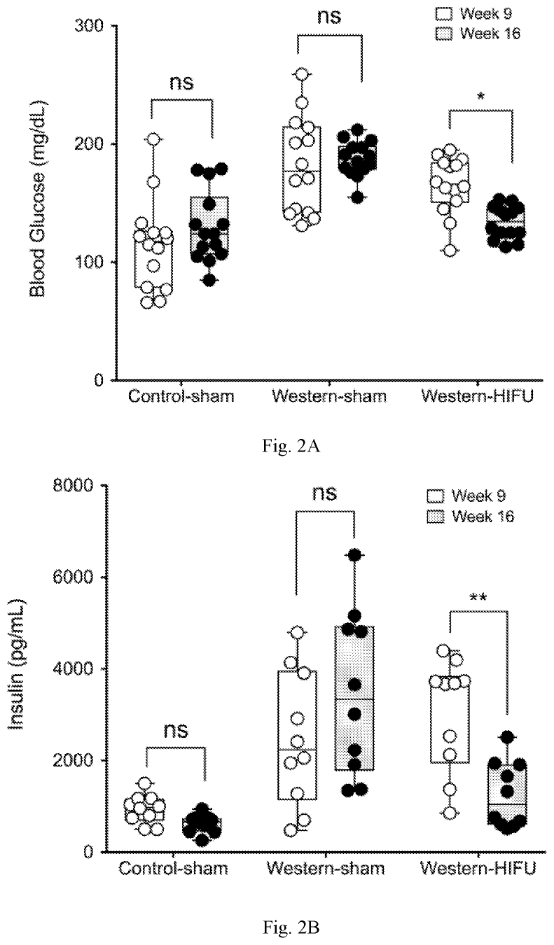

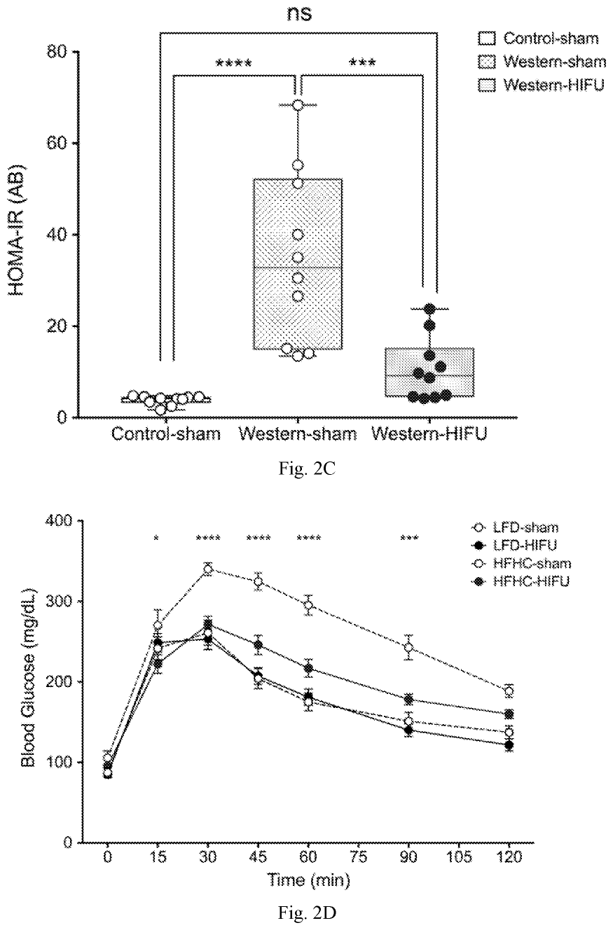

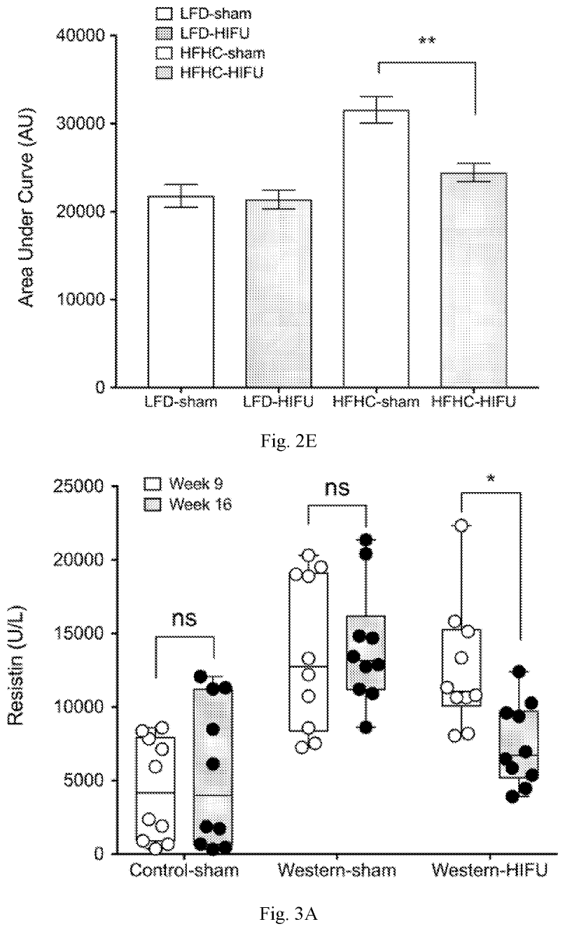

High Intensity Focused Ultrasound Lowers Fasting Blood Glucose and Insulin Levels, Improving Insulin Resistance and Glucose Tolerance in High-Fat High-Carbohydrate Fed Mice. Mice on the HFHC diet were assessed for their blood glucose levels at weeks 9 and 16. HFHC-sham mice did not change significantly between weeks 9 and 16. By contrast, mice in the HFHC-HIFU group had significantly reduced blood glucose levels between weeks 9 and 16 (FIG. 2A, p<0.01, 2-way ANOVA). Insulin levels were also measured between weeks 9 and 16. HFHC-sham mice did not demonstrate a change in their insulin levels while HFHC-HIFU mice demonstrated reduction in insulin (FIG. 2B, p<0.05, 2-way ANOVA). Applying the homeostatic model assessment insulin resistance (HOMA-IR) formula to data from week 16 revealed an increased insulin resistance in the HFHC-sham group when compared to the HFHC-HIFU group (FIG. 2C, p<0.001, Mann-Whitney). On week 16, the mice were subjected to a glucose tolerance test (GGT, 10% D Glucose solution, 1 g/kg), which revealed a significant improvement in glucose tolerance for the HFHC-HIFU mice in comparison to the HFHC-sham mice. The HFHC-HIFU group had a significantly lower peak glucose level (FIG. 2D, 30 min, p<0.0001, 2-way ANOVA), and a significantly lower area under the curve (FIG. 2E, p<0.001, 1-way ANOVA). In summary, mice in the HFHC-HIFU group have significantly reduced blood glucose and insulin levels between weeks 9 and 16, as well as, improved insulin resistance and glucose tolerance when compared to HFHC-sham mice.

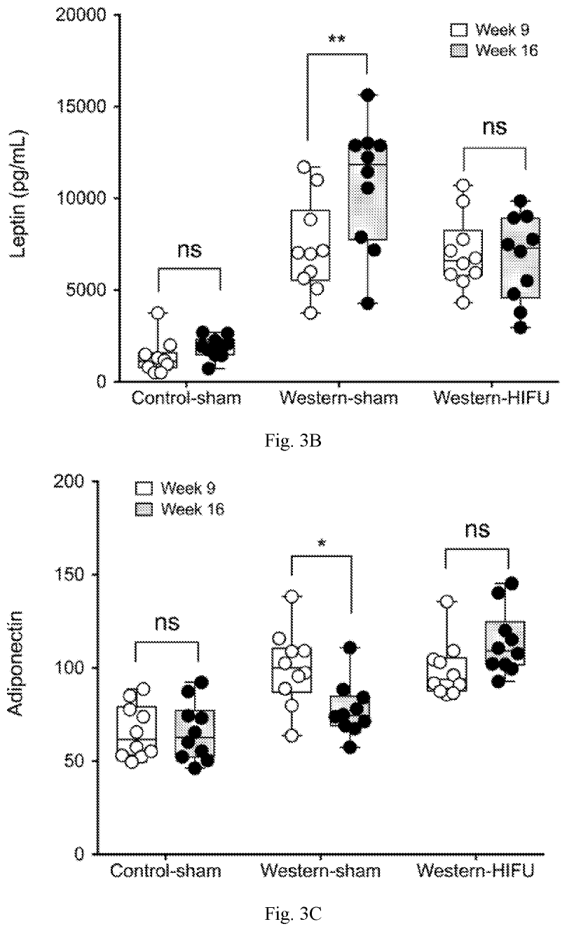

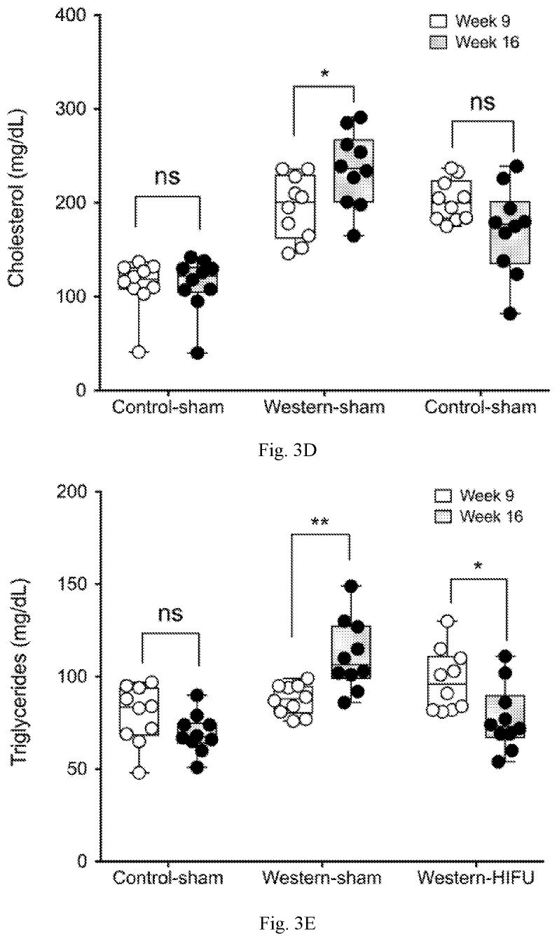

High Intensity Focused Ultrasound Alters Levels of Adipokines and Adipose Levels in High-Fat High-Carbohydrate Fed Mice. Adipokine levels for resistin, leptin, and adiponectin were evaluated for HFHC-fed mice between weeks 9 and 16. Resistin levels remained similar between weeks 9 and 16 for the HFHC-sham group, whereas resistin levels decreased in the HFHC-HIFU group (FIG. 3A, p<0.05, 2-way ANOVA). Leptin levels were shown to increase in HFHC-sham mice (FIG. 3B, p<0.05, 2-way ANOVA), while this increase was attenuated in HFHC-HIFU mice. Adiponectin levels were significantly reduced in HFHC-sham mice, while this decrease was not present in the HFHC-HIFU mice (FIG. 3C, p<0.05, 2-way ANOVA). Circulating levels of cholesterol and triglycerides were measured for the HFHC-fed mice between weeks 9 and 16. Mice in the HFHC-sham group were found to have elevated levels of cholesterol whereas the increase was attenuated in HFHC-HIFU mice (FIG. 3D, p<0.05, 2-way ANOVA). Triglyceride levels were revealed to increase significantly in HFHC-sham mice (FIG. 3E, p<0.01, 2-way ANOVA), while HIFU treatment significantly reduced triglyceride levels in HFHC-HIFU mice (FIG. 3E, p<0.05, 2-way ANOVA).

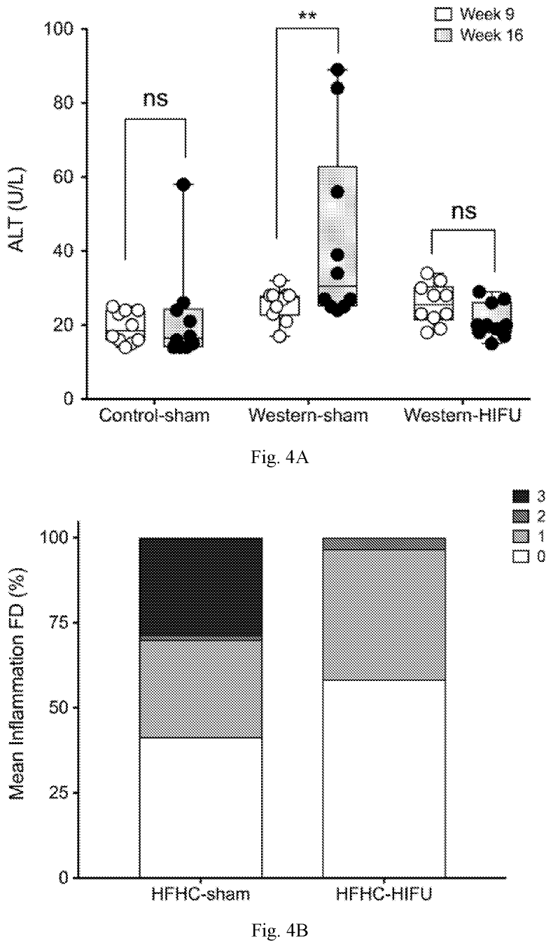

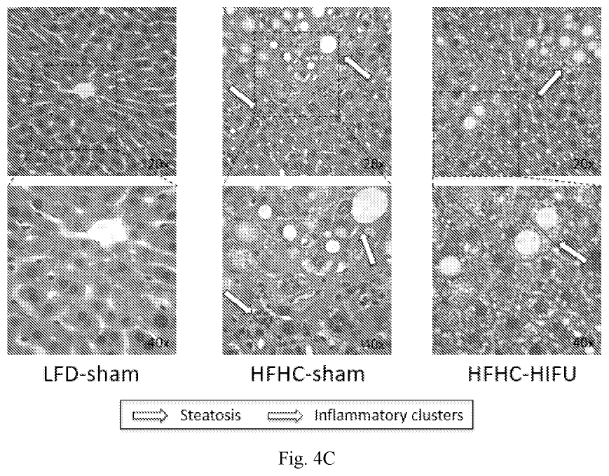

High Intensity Focused Ultrasound Attenuates Severity of Non Alcoholic Steatohepatitis Manifestations in High Fat High-Carbohydrate Fed Mice. Non-alcoholic Steatohepatitis (NASH) is associated with increased circulating levels of alanine aminotransferase (ALT) and inflammatory cell aggregation in the liver. At the end of the study (week 16), ALT levels were significantly increased in HFHC-sham mice. This increase was attenuated in the HFHC-HIFU mice (FIG. 4A, p<0.05, 2-way ANOVA). H&E stained liver sections of the HFHC fed mice revealed that the severity of inflammatory cell accumulation was lower in the HFHC-HIFU group than the HFHC-sham group (FIG. 4B). Representative Images for H&E stained sections of mouse livers are shown in FIG. 4C. Hepatic HIFU stimulation had no significant effect on aspartate aminotransferase levels. Asparate aminotransferase levels were evaluated in blood sampled HFHC diet fed animals between weeks 9 and 16. No significant difference was observed between weeks 9 and 16 for neither the HFHC-sham group nor the HFHC-HIFU group (not illustrated).

### REFERENCES

1. “Metabolic Syndrome.” National Heart Lung and Blood Institute. U.S. Department of Health and Human Services. Accessed Sep. 25, 2019. https://www.nhlbi.nih.gov/health-topics/metabolic-syndrome.
2. Samson, Susan L., and Alan J. Garber. “Metabolic syndrome.” Endocrinology and Metabolism Clinics 43, no. 1 (2014): 1-23.
3. Romeo, G. R., Lee, J., & Shoelson, S. E. (2012). Metabolic syndrome, insulin resistance, and roles of inflammation-mechanisms and therapeutic targets. Arteriosclerosis, thrombosis, and vascular biology, 32(8), 1771-1776.
4. Després, Jean-Pierre, and Isabelle Lemieux. “Abdominal obesity and metabolic syndrome.” Nature 444, no. 7121 (2006): 881.
5. Roberts, C. K., Hevener, A. L., & Barnard, R. J. (2013). Metabolic syndrome and insulin resistance: underlying causes and modification by exercise training. Comprehensive Physiology, 3(1), 1-58. doi:10.1002/cphy.c110062
6. Cornier M A, et al. (2008) The metabolic syndrome. Endocr. Rev. 29:777-822
7. di Biase, L., Falato, E., & Di Lazzaro, V. (2019). Transcranial Focused Ultrasound (tFUS) and Transcranial Unfocused Ultrasound (tUS) Neuromodulation: From Theoretical Principles to Stimulation Practices. Frontiers in Neurology, 10.
8. Cotero, V., Fan, Y., Tsaava, T., Kressel, A. M., Hancu, I., Fitzgerald, P., . . . & Kao, T. J. (2019). Noninvasive sub-organ ultrasound stimulation for targeted neuromodulation. Nature communications, 10(1), 952.

1. A method for one or more of treating metabolic syndrome, treating fatty liver disease, improving insulin resistance, treating inflammation and decreasing body weight in a subject in need thereof comprising applying ultrasound to the hepatic system of the subject in an amount effective to one or more of treat metabolic syndrome, treat fatty liver disease, improve insulin resistance, treat inflammation and decrease body weight.

2. The method of claim 1, wherein hepatic ultrasound reduces one or more of the subject's food intake, visceral fat accumulation and body weight.

3. The method of claim 1, wherein hepatic ultrasound does one or more of reduce blood glucose levels, reduce insulin levels, improve insulin resistance, and improve glucose tolerance in the subject.

4. The method of claim 1, wherein hepatic ultrasound reduces blood levels of one or more of resistin, leptin, cholesterol, triglyceride and alanine aminotransferase in the subject.

5. The method of claim 1, wherein hepatic ultrasound increases blood levels of adiponectin in the subject.

6. The method of claim 1, wherein the fatty liver disease is nonalcoholic fatty liver disease.

7. The method of claim 1, wherein the fatty liver disease is nonalcoholic steatohepatitis.

8. The method of claim 1, wherein hepatic ultrasound treats metabolic syndrome.

9. The method of claim 1, wherein the subject is on a high-fat, high-carbohydrate diet.

10. The method of claim 1, wherein the ultrasound is high intensity focused ultrasound.

11. The method of claim 1, wherein the ultrasound targets the porta hepatis.

12. The method of claim 1, wherein the subject is a human.
